(19) United States
(12) Patent Application Publication
(10) Pub. No.: US 2026/0273120 A1
(43) Pub. Date: Sep. 17, 2026

(54) ORAL UTENSIL DISINFECTION BOX

(71) Applicant: Thumbs Up Innovations Technology Co., Ltd., Zhuhai (CN)

(72) Inventors: JIACHENG HUANG, Zhuhai (CN); LILIN XU, Zhuhai (CN); XIU ZOU, Zhuhai (CN)

(21) Appl. No.: 19/077,488

(22) Filed: Mar. 12, 2025

(51) Int. Cl. A61L 2/26; A61L 2/24; F26B 9/00; F26B 9/06

(52) U.S. Cl. CPC A61L 2/26; A61L 2/24; F26B 9/003; F26B 9/06; A61L 2103/15; A61L 2202/121; A61L 2202/14

(57) ABSTRACT

An oral utensil disinfection box includes a main body, a box cover, a control module, and a sensing unit for sensing or recognition. The main body is provided with a disinfection cavity with a first opening on one side. The main body is equipped with a driving unit and a sterilization and disinfection unit. The sterilization and disinfection unit is used for sterilizing and disinfecting an oral utensil. The box cover is connected to the driving unit. The sensing unit, the sterilization and disinfection unit, and the driving unit are all electrically connected to the control module. The box cover covers the first opening when in a closed state. The control module is configured for driving the driving unit to work based on an electrical signal fed back by the sensing unit, so that the driving unit drives the box cover to move to open or close the first opening.

### TECHNICAL FIELD

The present invention relates to the technical field of disinfection boxes, particularly to an oral utensil disinfection box.

### BACKGROUND ART

A toothbrush is a tool used by people to clean their teeth. Due to the moist state of a toothbrush head after use, it is prone to mold and bacterial growth. Therefore, disinfection boxes have emerged on the market for disinfecting and sterilizing the toothbrush head. At present, in order to play a dustproof role for the toothbrush head, the existing toothbrush head disinfection boxes generally have a flip cover on a storage cavity that accommodates the toothbrush head. When users place or remove the toothbrush head from the storage cavity, the users usually need to manually open the flip cover. During this process, fingers are prone to touch the bristles of the toothbrush head, thereby causing secondary pollution to the toothbrush head. Moreover, manual operation is required, which is inconvenient for users to use.

Furthermore, the toothbrush head is in a humid environment after use, which is easy to mold and breed bacteria. However, the existing toothbrush head disinfection boxes generally use only UV lamps to achieve sterilization and disinfection, and cannot dry the wet toothbrush head, resulting in a single function.

### SUMMARY

The main purpose of the present invention is to provide an oral utensil disinfection box, so as to solve the problem of inconvenient use for a user and secondary pollution of a toothbrush head caused by a flip cover on an existing toothbrush head disinfection box needing to be manually operated by the user.

In order to solve the above technical problem, a technical solution provided in the present invention is as follows.

An oral utensil disinfection box includes a main body, a box cover, a control module, and a sensing unit for sensing or recognition. The main body is provided with a disinfection cavity. The disinfection cavity is provided with a first opening on one side and is used for placement of an oral utensil. The main body is equipped with a driving unit and a sterilization and disinfection unit. The sterilization and disinfection unit is used for sterilizing and disinfecting the oral utensil.

The box cover is connected to the driving unit. The sensing unit, the sterilization and disinfection unit, and the driving unit are all electrically connected to the control module. The box cover covers the first opening when in a closed state. The control module is configured for driving the driving unit to work based on an electrical signal fed back by the sensing unit, so that the driving unit drives the box cover to move to open or close the first opening.

The present invention has the following beneficial effects. Compared with the prior art, in this embodiment, the sensing unit and the driving unit are configured on the disinfection box, and the driving unit is used for driving the box cover to move. The sensing unit is used for feeding back the electrical signal to the control module when the sensing unit senses a user or an object approaching. When the control module receives the electrical signal, the control module drives the driving unit to work to drive the box cover to move to open or close the first opening, so that the user can place the oral utensil into the disinfection cavity or remove the oral utensil from the disinfection cavity, thereby achieving automatic opening or closing of the box cover. No manual operation is required to open or close the box cover, making it convenient for the user to use. This solves the problem of inconvenient use caused by the flip cover on the existing oral utensil disinfection box needing to be manually operated by the user. In this way, the likelihood of the oral utensil, such as a toothbrush head or a nozzle, coming into contact with a hand or the flip cover can also be effectively reduced, thereby effectively reducing the secondary pollution to the oral utensil.

### BRIEF DESCRIPTION OF THE DRAWINGS

Implementations of the present disclosure will now be described, by way of embodiment, with reference to the attached figures. It should be understood, the drawings are shown for illustrative purpose only, for ordinary person skilled in the art, other drawings obtained from these drawings without paying creative labor by an ordinary person skilled in the art should be within scope of the present disclosure.

FIG. 1 is a perspective view of the present invention.

FIG. 2 is a perspective view of the present invention from another angle of view.

FIG. 3 is a schematic diagram of the present invention with a box cover removed.

FIG. 4 is an exploded view of the present invention.

FIG. 5 is an exploded view of the present invention from another angle of view.

FIG. 6 is a sectional view cut along a central axis of a circular column when a front shell, a box cover, and a first position limiting member of the present invention are assembled together.

FIG. 7 is an enlarged view of area A in FIG. 6.

FIG. 8 is a schematic diagram with a front shell, a box cover, and a first position limiting member of the present invention assembled together.

FIG. 9 is a structural diagram of a first position limiting member of the present invention.

FIG. 10 is a schematic diagram with a bracket, an air blowing unit, and a driving unit of the present invention assembled together.

FIG. 11 is an exploded view of a bracket of the present invention.

FIG. 12 is a perspective view of a box cover of the present invention.

FIG. 13 is an exploded view of a box cover of the present invention.

### DETAILED DESCRIPTION OF THE EMBODIMENTS

It will be appreciated that for simplicity and clarity of illustration, where appropriate, reference numerals have been repeated among the different figures to indicate corresponding or analogous elements. In addition, numerous specific details are set forth in order to provide a thorough understanding of the exemplary embodiments described herein. However, it will be understood by those of ordinary skill in the art that the exemplary embodiments described herein may be practiced without these specific details. In other instances, methods, procedures, and components have not been described in detail so as not to obscure the related relevant feature being described. Also, the description is not to be considered as limiting the scope of the exemplary embodiments described herein. The drawings are not necessarily to scale and the proportions of certain parts may be exaggerated to better illustrate details and features of the present disclosure.

The term “comprising” when utilized, means “including, but not necessarily limited to”; it specifically indicates open-ended inclusion or membership in the so-described combination, group, series, and the like. The disclosure is illustrated by way of example and not by way of limitation in the figures of the accompanying drawings in which like references indicate similar elements. It should be noted that references to “an” or “one” embodiment in this disclosure are not necessarily to the same embodiment, and such references can mean “at least one”. In addition, the terms “first” and “second” are used for descriptive purposes only and cannot be understood as indicating or implying relative importance or implying the number of indicated technical features. Thus, the features defined as “first” and “second” may explicitly or implicitly include one or more of the features. In the description of embodiments of the application, “a plurality of” means two or more, unless otherwise specifically defined.

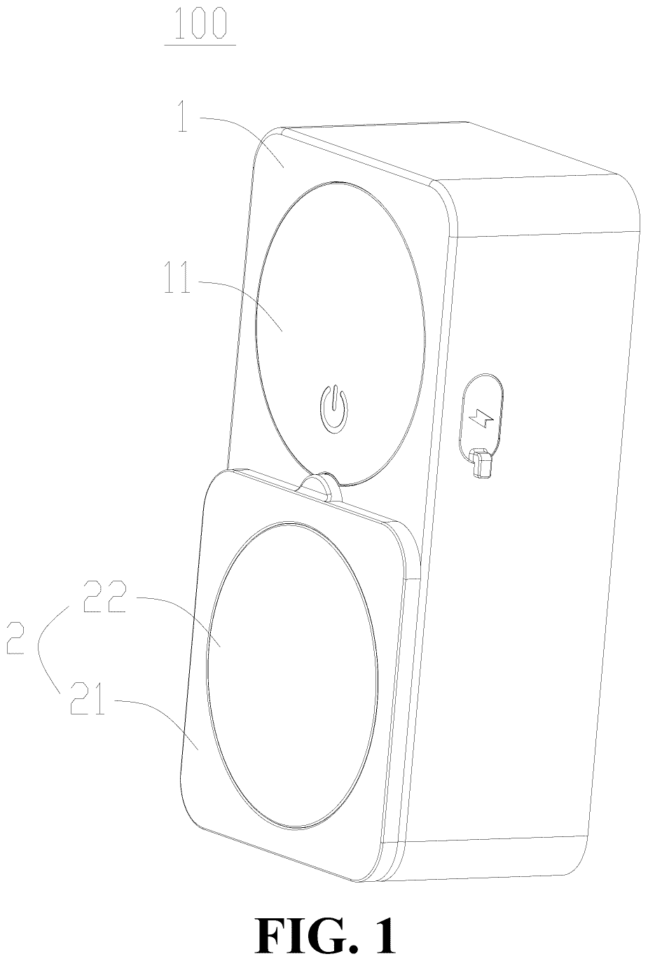

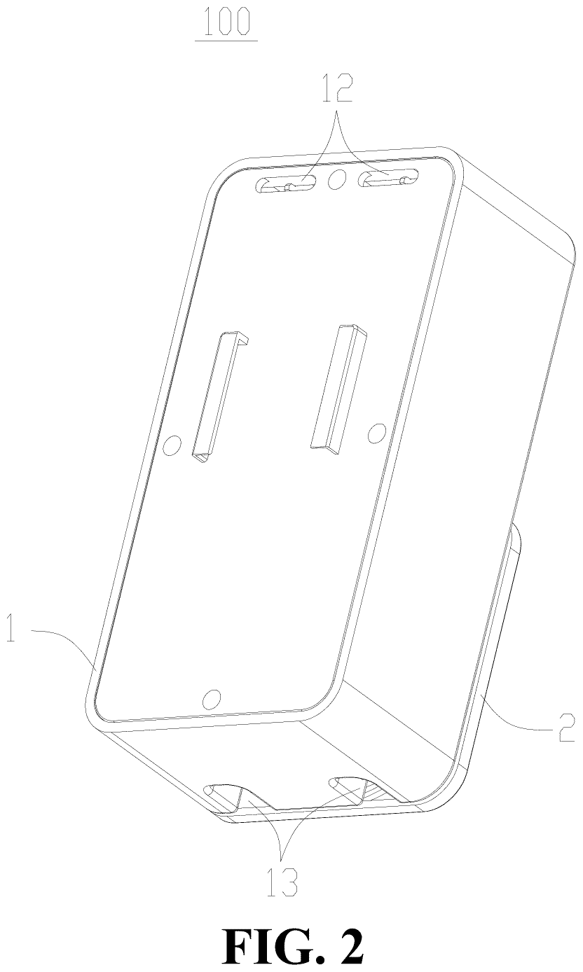

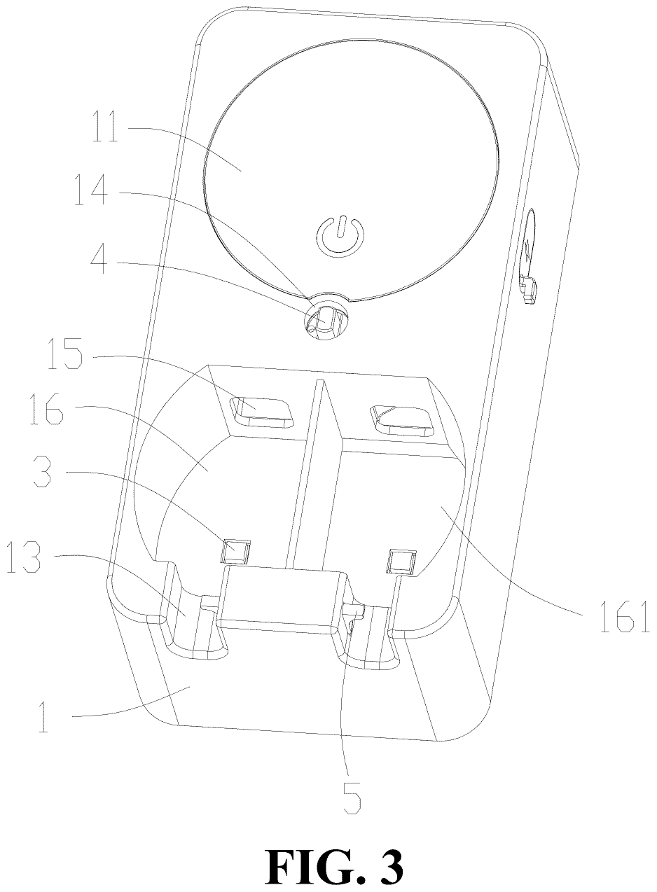

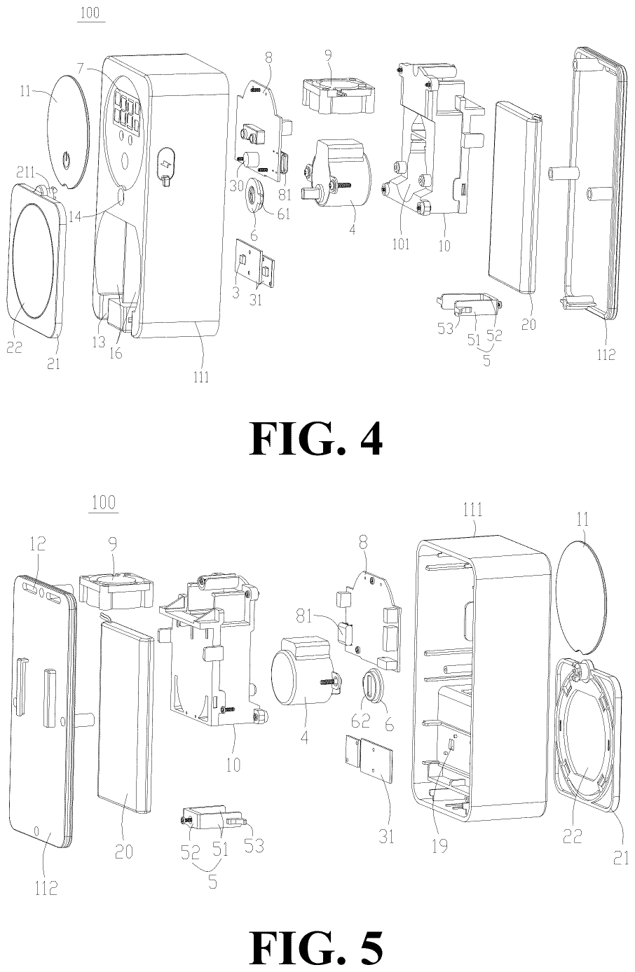

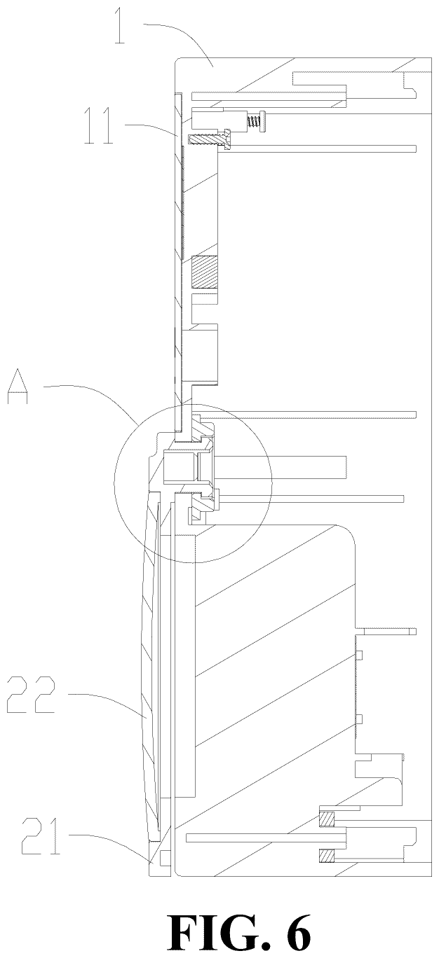

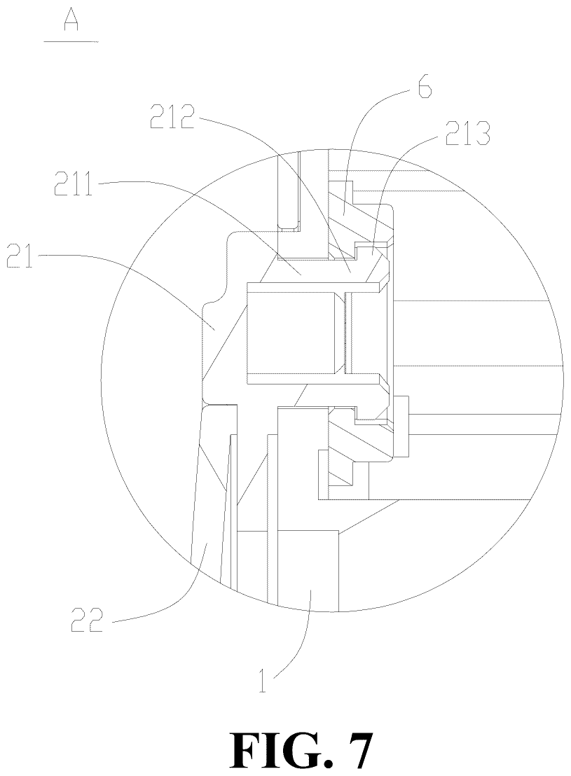

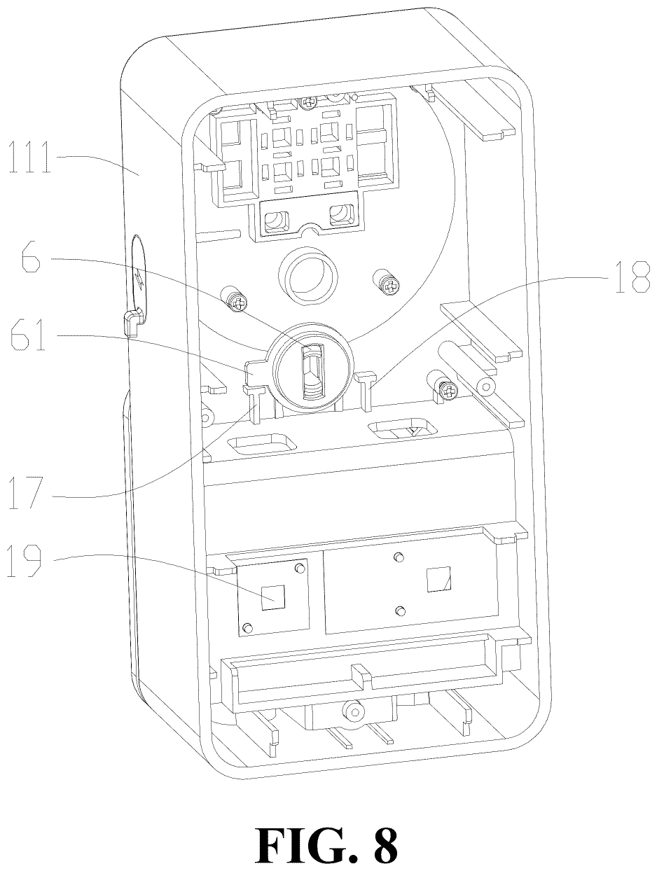

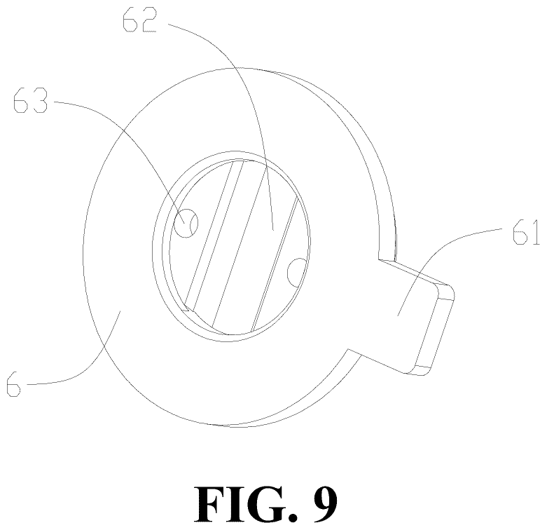

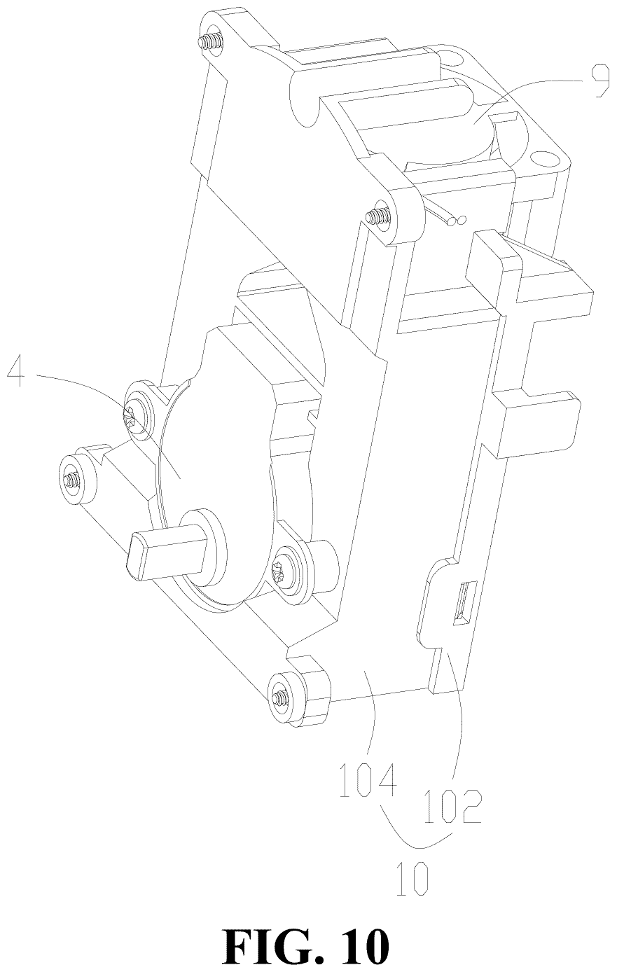

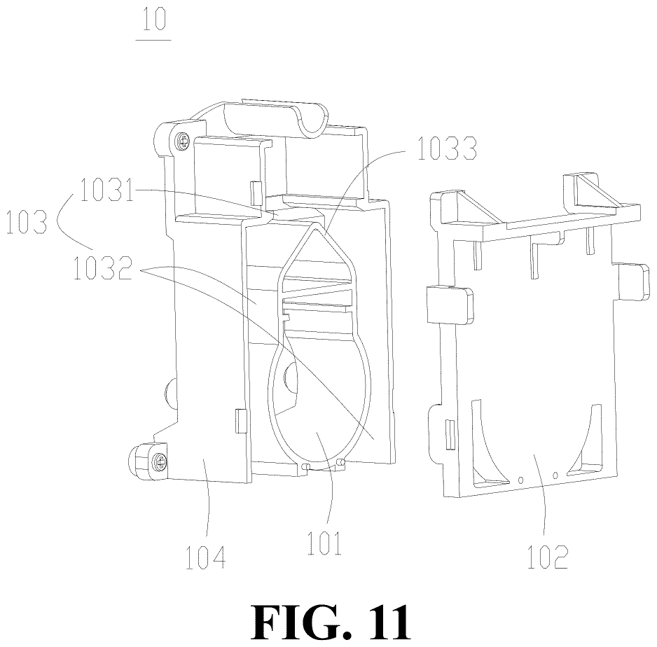

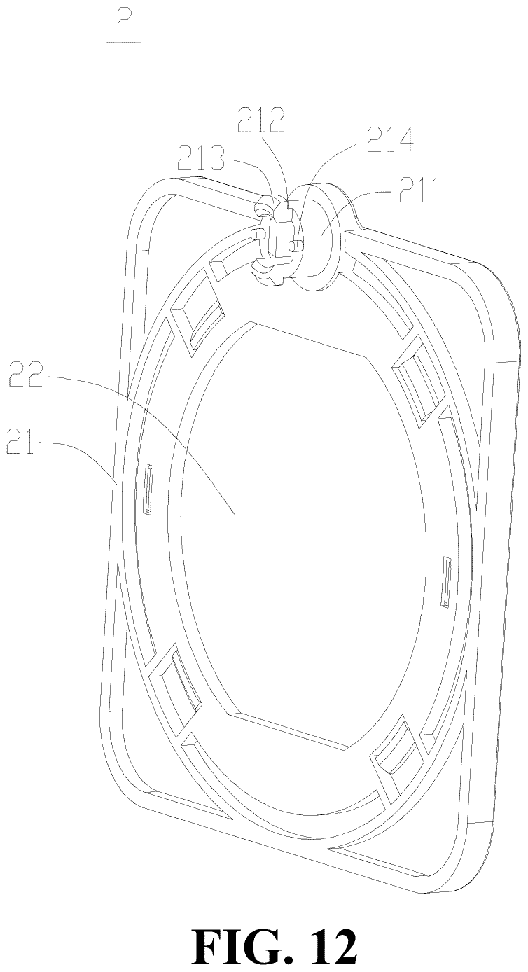

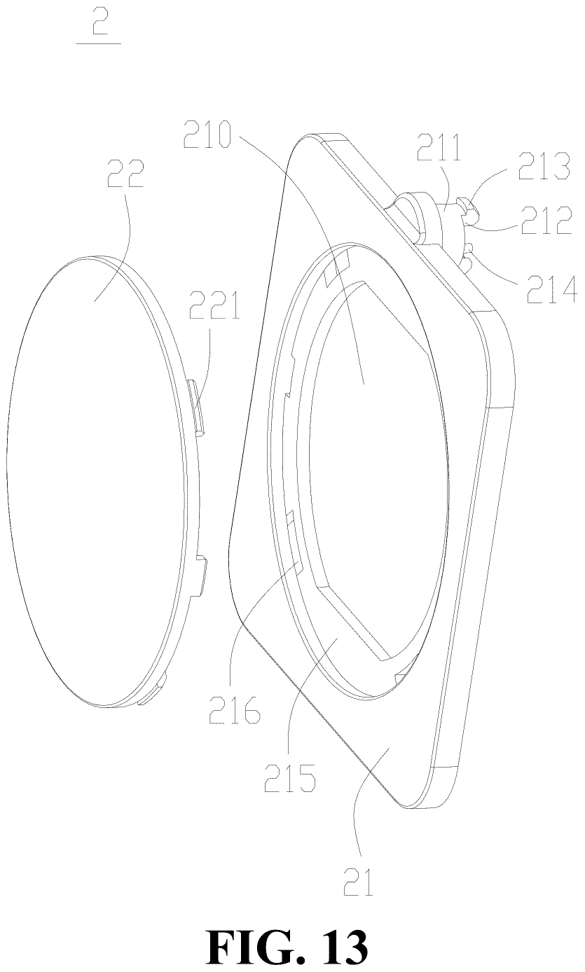

FIGS. 1-13 show an oral utensil disinfection box 100 in an embodiment of the present invention.

The oral utensil disinfection box 100 includes a main body 1, a box cover 2, a control module 8, and a sensing unit 30 for sensing or recognition. The main body 1 is provided with a disinfection cavity 16. The disinfection cavity 16 is provided with a first opening 161 on one side and is used for placement of an oral utensil. The main body 1 is equipped with a driving unit 4 and a sterilization and disinfection unit 3. The sterilization and disinfection unit 3 is used for sterilizing and disinfecting the oral utensil. The box cover 2 is connected to the driving unit 4. The sensing unit 30, the sterilization and disinfection unit 3, and the driving unit 4 are all electrically connected to the control module 8. The box cover 2 covers the first opening 161 when in a closed state. The control module 8 is configured for driving the driving unit 4 to work based on an electrical signal fed back by the sensing unit 30, so that the driving unit 4 drives the box cover 2 to move to open or close the first opening 161.

In this embodiment, the sensing unit 30 and the driving unit 4 are configured on the disinfection box 100, and the driving unit 4 is used for driving the box cover 2 to move. The sensing unit 30 is used for feeding back the electrical signal to the control module 8 when the sensing unit 30 senses a user or an object approaching. When the control module 8 receives the electrical signal, the control module 8 drives the driving unit 4 to work to drive the box cover 2 to move to open or close the first opening 161, so that the user can place the oral utensil into the disinfection cavity 16 or remove the oral utensil from the disinfection cavity 16, thereby achieving automatic opening or closing of the box cover 2. This solves the problem of inconvenient use caused by a flip cover on an existing oral utensil disinfection box needing to be manually operated by the user. In this way, the likelihood of the oral utensil, such as a toothbrush head or a tooth washer nozzle, coming into contact with a hand or the flip cover can also be effectively reduced, thereby effectively reducing the secondary pollution to the oral utensil.

Specifically, the oral utensil can be a toothbrush head, a toothbrush, a mouth mirror, and a nozzle of a tooth washer, etc. The control module 8 can be disposed on the main body 1, or can also be disposed outside the main body 1, that is, functioning as an independent controller. The sensing unit 30 can be disposed on the main body 1 or outside the main body 1.

The sterilization and disinfection unit 3 can be an ultraviolet lamp, a UV lamp, or an ozone disinfection lamp, etc. The oral utensil disinfection box 100 can be fixed to a wall, a cabinet, and other positions by a bracket 10, a screw, pasting, and other methods. The sensing unit 30 can be a distance sensor, an infrared sensor, an infrared human body sensor, a camera recognition module, an iris recognition module, etc. When the user approaches the disinfection box 100 in this embodiment, the sensing unit 30 detects that an object is approaching, a human body is approaching, or a human face, an iris, etc., are successfully recognized, then the sensing unit 30 can feed back an electrical signal to the control module 8, and the control module 8 drives the driving unit 4 to work according to the electrical signal to drive the box cover 2 to move to open or close the first opening 161.

The movement of the box cover 2 can be rotating or sliding. When the movement of the box cover 2 is rotating, the driving unit 4 can be a motor. When the movement of the box cover 2 is sliding, the driving unit 4 can be a linear module, a motor matching rack or a connecting rod, or the driving unit 4 can also be a cylinder and other structures.

In one embodiment, the box cover 2 is provided with a position limiting protrusion 61. When the driving unit 4 is driven and drives the box cover 2 to move, the position limiting protrusion 61 moves together. The main body 1 is provided with an open position limiting portion 18 and a closed position limiting portion 17 beside the position limiting protrusion 61. The position limiting protrusion 61 abuts against the closed position limiting portion 17 when the box cover 2 is in the closed state. The position limiting protrusion 61 also abuts against the open position limiting portion 18 when the box cover 2 is in an open state. By using the open position limiting portion 18 and the closed position limiting portion 17 to limit the motion displacement of the position limiting protrusion 61, the motion displacement of the box cover 2 during movement can be restricted, so that the box cover 2 can cover the first opening 161 in the closed state and open the first opening 161 in the open state, making it convenient for the user to use.

In one embodiment, the oral utensil disinfection box 100 further includes a first position limiting member 6. The box cover 2 is connected to the first position limiting member 6. The first position limiting member 6 is arranged inside the box cover 2. The position limiting protrusion 61 is positioned on the first position limiting member 6. The driving unit 4 is a motor, that is, the box cover 2 is driven to rotate when a rotating shaft of the motor rotates, and the first position limiting member 6 is driven to rotate together when the box cover 2 rotates. By using the open position limiting portion 18 and the closed position limiting portion 17 to cooperate with the position limiting protrusion 61, an angle of rotation of the box cover 2 can be limited.

In one embodiment, a central through hole 14 is defined in a surface of the main body 1. The box cover 2 is provided with a circular column 211 on a surface close to the main body 1. The circular column 211 is connected to the driving unit 4. The circular column 211 is provided with a plurality of spaced connecting portions 212 on one side away from the box cover 2. The connecting portion 212 is provided with a first engaging block 213. The first position limiting member 6 is provided with a first engaging opening 62 used in cooperation with the first engaging block 213. The plurality of connecting portions 212 are placed in the first engaging opening 62, and the first engaging block 213 is positioned on one side of the first position limiting member 6 away from the box cover 2, so that the box cover 2 can be fixedly connected to the first position limiting member 6, and the box cover 2 and the first position limiting member 6 are rotatably arranged on the main body 1. According to this structure, slots can also be formed between the plurality of first engaging blocks 213 for the installation of the rotating shaft of the motor, so that the rotating shaft of the motor can be connected to the box cover 2. Moreover, the plurality of spaced connecting portions 212 can allow the connecting portions 212 to have a certain degree of elasticity, so that when the first engaging block 213 passes through the first engaging opening 62, the plurality of first engaging blocks 213 can be provided with a deformation elastic force, thereby enabling the first engaging block 213 to pass through the first engaging opening 62. The circular column 211 is provided with a positioning protrusion 214 on one side away from the box cover 2. A positioning slot 63 is defined in the first position limiting member 6. The positioning protrusion 214 is placed in the positioning slot 63 to enable the box cover 2 and the first position limiting member 6 to rotate together when the motor is driven.

Of course, in other embodiments, the first position limiting member 6 can also be fixed to the circular column 211 by screws, which can also enable the box cover 2 and the first position limiting member 6 to rotate together.

In one embodiment, a total number of the disinfection cavities 16 may be one. In other embodiments, the total number of the disinfection cavities 16 may be multiple, and the disinfection cavities 16 are arranged at intervals. When the box cover 2 is in the closed state, the box cover 2 is capable of covering the first openings 161 of the plurality of disinfection cavities 16, so that automatic opening and closing of the box cover 2 can be achieved by configuring one driving unit 4. Of course, in other embodiments, the total number of the disinfection cavities 16 may be multiple. The box cover 2, the disinfection cavity 16, the driving unit 4, and the sensing unit 30 are consistent in number, that is, each driving unit 4 corresponds to one sensing unit 30, one box cover 2, and one disinfection cavity 16 for use.

In one embodiment, the oral utensil disinfection box 100 further includes an air blowing unit 9 for drying the oral utensil. The air blowing unit 9 is electrically connected to the control module 8. The main body 1 is provided with air inlet holes 12. The air inlet hole 12 is connected to an air inlet opening of the air blowing unit 9, and an air outlet opening of the air blowing unit 9 is in communication with the disinfection cavity 16. By configuring the air blowing unit 9, when the control module 8 drives the air blowing unit 9, the air blowing unit 9 generates an airflow, so that the airflow is sent out from the air outlet opening of the air blowing unit 9 and then enters the disinfection cavity 16, so as to dry the oral utensil and prevent the problem of bacteria breeding easily when the oral utensil is in a damp state.

Specifically, the air blowing unit 9 can be a fan or a blower, etc.

In one embodiment, an air duct 103 is defined in the main body 1. The air blowing unit 9 is arranged on the main body 1. A first end of the air duct 103 is in communication with the air outlet opening of the air blowing unit 9, and a second end of the air duct 103 is in communication with the disinfection cavity 16. The air blowing unit 9 generates an airflow and transmits the airflow to the disinfection cavity 16 through the air duct 103.

In one embodiment, a total number of the disinfection cavities 16 is multiple, and the disinfection cavities 16 are arranged at intervals. The air duct 103 includes a first hole segment 1031 and a plurality of second hole segments 1032. The plurality of second hole segments 1032 are respectively in communication with the first hole segment 1031. Each of the second hole segments 1032 is in communication with one disinfection cavity 16 at a position away from the first hole segment 1031. The air blowing unit 9 generates an airflow, and the airflow passes through the first hole segment 1031 and the second hole segment 1032 in sequence and then enters the disinfection cavity 16, so that one air blowing unit 9 can dry the oral utensils in the plurality of disinfection cavities 16.

In one embodiment, a bracket 10 is provided inside the main body 1. The air blowing unit 9, the driving unit 4, and the air duct 103 are all arranged on the bracket 10, so as to reasonably and effectively configure the air blowing unit 9, the driving unit 4, and the air duct 103, making the volume of the oral utensil disinfection box 100 in this embodiment smaller. The bracket 10 is equipped with an air guide portion 1033 between two adjacent second hole segments 1032. A width of the air guide portion 1033 gradually decreases along a direction close to the first hole segment 1031, that is, the air guide portion 1033 is in an inverted “V” shape in uppercase, so that the airflow generated by the air blowing unit 9 passes through the first hole segment 1031 and then is effectively distributed to each of the second hole segments 1032 through the air guide portion 1033, which can play a role in guiding air.

In one embodiment, a first mounting cavity 101 is defined in a surface of the bracket 10. A total number of the second hole segments 1032 is two. The first mounting cavity 101 is positioned between the two second hole segments 1032, and the driving unit 4 is at least partially arranged in the first mounting cavity 101, thereby effectively utilizing a space on the bracket 10, making the volume of the oral utensil disinfection box 100 of this embodiment smaller, and making the structure of the oral utensil disinfection box 100 of this embodiment more compact and stable.

Specifically, in order to facilitate production and manufacturing of the bracket 10, the bracket 10 includes a first shell 104 and a second shell 102. The second shell 102 is connected to the first shell 104 to form the air duct 103 and the first mounting cavity 101. The first shell 104 and the second shell 102 are both positioned inside the main body 1. The disinfection cavity 16 is positioned below the bracket 10. The main body 1 can be formed by fixedly connecting a face shell 111 and a bottom shell 112 in a manner of screws, buckles, ultrasonic welding, etc. The first shell 104 and the second shell 102 can be fixedly connected by screws, buckles, ultrasonic welding, etc. Moreover, the first shell 104 or the second shell 102 is fixed to the main body 1 to achieve the fixation of the bracket 10 inside the main body 1.

In one embodiment, the oral utensil disinfection box 100 further includes a battery 20 electrically connected to the control module 8. The control module 8 is a circuit board. The main body 1 is provided with a charging interface 81 for connecting to an external power supply. The external power supply is connected through the charging interface 81, the connected power supply is processed by the control module 8, and thereafter the battery 20 is charged, thereby extending the usage time of the oral utensil disinfection box 100 in this embodiment.

Of course, in other embodiments, the battery 20 can also be a non-rechargeable battery 20, that is, in a replaceable form, which can also extend the usage time of the oral utensil disinfection box 100 in this embodiment.

In one embodiment, the circuit board is positioned above the disinfection cavity 16, and the battery 20 is positioned on one side of the air duct 103 away from the circuit board, thereby reasonably distributing the space inside the main body 1, and further reducing the volume of the oral utensil disinfection box 100 in this embodiment.

In one embodiment, the oral utensil disinfection box 100 further includes a display 7 electrically connected to the control module 8. The display 7 is positioned on one side of the air duct 103 away from the battery 20. The main body 1 is provided with a first transparent cover plate 11 at a position corresponding to the display 7. The display 7 is used for displaying a battery level of the battery 20. A power output end of the driving unit 4 is positioned between the first transparent cover plate 11 and the disinfection cavity 16.

Specifically, the display 7 can be a digital display 7, an LCD display screen, or a touch display screen.

In one embodiment, the sensing unit 30 is arranged on the circuit board and positioned below the display 7. Of course, in other embodiments, the sensing unit 30 can also be placed on the main body 1 and not below the display 7, and can be placed on a left side, a right side, or an upper side of the display 7.

In one embodiment, the oral utensil disinfection box 100 further includes an adapter plate 31 arranged inside the main body 1. The sterilization and disinfection unit 3 is arranged on the adapter plate 31. A total number of the disinfection cavities 16 is multiple. The sterilization and disinfection units 3 and the disinfection cavities 16 are consistent in number, so as to fix the sterilization and disinfection units 3. The main body 1 is provided with a second opening 19 at a position corresponding to the disinfection cavity 16. Each of the sterilization and disinfection units 3 is placed in the second opening 19 on each of the disinfection cavities 16, so as to achieve sterilization and disinfection of the oral utensil in each of the disinfection cavities 16.

In one embodiment, when the oral utensil is a toothbrush head or a toothbrush, a bottom portion of the main body 1 is provided with a hanging opening 13 at a position corresponding to the disinfection cavity 16 for avoiding a toothbrush handle. The main body 1 is provided with an elastic clamping block 53 on a side wall of the hanging opening 13. The elastic clamping block 53 has elasticity and is used for limiting the toothbrush handle at the hanging opening 13. Specifically, the hanging opening 13 is adapted to the toothbrush handle, and the elastic clamping block 53 can be used for limiting a position of the toothbrush handle when the toothbrush handle is placed inside the hanging opening 13, thereby preventing the toothbrush head from detaching from the disinfection cavity 16 when the box cover 2 is not closed. Moreover, the elastic clamping block 53 has elasticity, making it easy for the toothbrush handle to be placed in the hanging opening 13 or removed from the hanging opening 13.

In one embodiment, the oral utensil disinfection box 100 further includes a U-shaped second position limiting member 5. The second position limiting member 5 is positioned below the disinfection cavity 16. The disinfection cavities 16 and the hanging openings 13 are both two in number. The second position limiting member 5 includes a connecting rod 52 and two clamping rods 51. The connecting rod 52 is arranged on the main body 1. The two clamping rods 51 are connected to the connecting rod 52 and are arranged at intervals. The two clamping rods 51 are positioned between the two hanging openings 13. Each of the clamping rods 51 is equipped with the elastic clamping block 53, so that the elastic clamping block 53 has a certain degree of elasticity. The elastic clamping block 53 is partially placed inside the hanging opening 13 in an initial state to adapt to the toothbrush handle for use. Of course, in other embodiments, the elastic clamping block 53 with elasticity can also be made of soft rubber material or be used in conjunction with springs to achieve a certain degree of deformability and restore to the initial state after deformation.

In one embodiment, the box cover 2 includes a cover body 21 and a second transparent cover plate 22. The second transparent cover plate 22 is provided on the cover body 21 for a user to observe the disinfection cavity 16. The second transparent cover plate 22 corresponds to a position of the first opening 161 when the box cover 2 is in the closed state, so that the user can view the oral utensil inside the disinfection cavity 16.

In one embodiment, a third opening 210 is defined in the cover body 21. A surface of the cover body 21 is recessed at a periphery corresponding to the third opening 210 to form an embedding slot 215. The cover body 21 is provided with a second engaging opening 216 on a bottom surface corresponding to the embedding slot 215. The second transparent cover plate 22 is provided with a second engaging block 221. The second engaging block 221 is engaged with the second engaging opening 216 to fix the second transparent cover plate 22 on the cover body 21. Of course, in other embodiments, the second transparent cover plate 22 can also be fixed to the cover body 21 by ultrasonic welding, pasting, or other methods.

The main body 1 can also be equipped with a detection unit for detecting the closing or opening of the box cover 2. The detection unit is electrically connected to the control module 8, so that after the box cover 2 is closed, the control module 8 receives an electrical signal from the detection unit to drive the sterilization and disinfection unit 3 to disinfect and drive the air blowing unit 9 to blow air. When the box cover 2 is opened, the control module 8 stops driving the sterilization and disinfection unit 3 to disinfect and stops driving the air blowing unit 9 to blow air. Specifically, the detection unit can be an infrared sensor, a distance measuring sensor, etc. Of course, in other embodiments, the main body 1 can also be equipped with a functional button electrically connected to the control module 8, such as a push switch, a dip switch, a touch switch, etc. By using the functional button, the control module 8 can turn on or off the sterilization and disinfection unit 3 to disinfect and/or the air blowing unit 9 to blow air.

The above description only describes embodiments of the present disclosure, and is not intended to limit the present disclosure; various modifications and changes can be made to the present disclosure. Any modifications, equivalent substitutions, and improvements made within the spirit and scope of the present disclosure are intended to be included within the scope of the present disclosure.

What is claimed is:

1. An oral utensil disinfection box, comprising:
a main body;
a box cover;
a control module; and
a sensing unit for sensing or recognition;
wherein the main body is provided with a disinfection cavity, the disinfection cavity is provided with a first opening on one side and is used for placement of an oral utensil, the main body is equipped with a driving unit and a sterilization and disinfection unit, and the sterilization and disinfection unit is used for sterilizing and disinfecting the oral utensil;
wherein the box cover is connected to the driving unit; the sensing unit, the sterilization and disinfection unit, and the driving unit are all electrically connected to the control module; the box cover covers the first opening when in a closed state; and the control module is configured for driving the driving unit to work based on an electrical signal fed back by the sensing unit, so that the driving unit drives the box cover to move to open or close the first opening.

2. The oral utensil disinfection box according to claim 1, wherein the box cover is provided with a position limiting protrusion; when the driving unit is driven and drives the box cover to move, the position limiting protrusion moves together; the main body is provided with an open position limiting portion and a closed position limiting portion beside the position limiting protrusion; the position limiting protrusion abuts against the closed position limiting portion when the box cover is in the closed state; and the position limiting protrusion also abuts against the open position limiting portion when the box cover is in an open state.

3. The oral utensil disinfection box according to claim 2, further comprising a first position limiting member, wherein the driving unit is a motor, the box cover is connected to the first position limiting member, the first position limiting member is arranged inside the box cover, and the position limiting protrusion is positioned on the first position limiting member.

4. The oral utensil disinfection box according to claim 3, wherein a central through hole is defined in a surface of the main body, the box cover is provided with a circular column on a surface close to the main body, the circular column is connected to the driving unit, the circular column is provided with a plurality of spaced connecting portions on one side away from the box cover, the connecting portion is provided with a first engaging block, the first position limiting member is provided with a first engaging opening used in cooperation with the first engaging block, the plurality of connecting portions are placed in the first engaging opening, and the first engaging block is positioned on one side of the first position limiting member away from the box cover; the circular column is provided with a positioning protrusion on one side away from the box cover, a positioning slot is defined in the first position limiting member, and the positioning protrusion is placed in the positioning slot.

5. The oral utensil disinfection box according to claim 1, wherein a total number of the disinfection cavities is multiple, and the disinfection cavities are arranged at intervals; and when the box cover is in the closed state, the box cover is capable of covering the first openings of the plurality of disinfection cavities.

6. The oral utensil disinfection box according to claim 1, further comprising an air blowing unit for drying the oral utensil, wherein the air blowing unit is electrically connected to the control module, the main body is provided with air inlet holes, the air inlet hole is connected to an air inlet opening of the air blowing unit, and an air outlet opening of the air blowing unit is in communication with the disinfection cavity.

7. The oral utensil disinfection box according to claim 6, wherein an air duct is defined in the main body, the air blowing unit is arranged on the main body, a first end of the air duct is in communication with the air outlet opening of the air blowing unit, and a second end of the air duct is in communication with the disinfection cavity.

8. The oral utensil disinfection box according to claim 7, wherein a total number of the disinfection cavities is multiple, and the disinfection cavities are arranged at intervals;
the air duct comprises a first hole segment and a plurality of second hole segments, the plurality of second hole segments are respectively in communication with the first hole segment, and each of the second hole segments is in communication with one disinfection cavity at a position away from the first hole segment.

9. The oral utensil disinfection box according to claim 8, wherein a bracket is provided inside the main body; the air blowing unit, the driving unit, and the air duct are all arranged on the bracket; the bracket is equipped with an air guide portion between two adjacent second hole segments; and a width of the air guide portion gradually decreases along a direction close to the first hole segment.

10. The oral utensil disinfection box according to claim 9, wherein a first mounting cavity is defined in a surface of the bracket, a total number of the second hole segments is two, the first mounting cavity is positioned between the two second hole segments, and the driving unit is at least partially arranged in the first mounting cavity.

11. The oral utensil disinfection box according to claim 9, wherein the bracket comprises a first shell and a second shell, the second shell is connected to the first shell to form the air duct and a first mounting cavity, the first shell and the second shell are both positioned inside the main body, and the disinfection cavity is positioned below the bracket.

12. The oral utensil disinfection box according to claim 7, further comprising a battery electrically connected to the control module, wherein the control module is a circuit board, and the main body is provided with a charging interface for connecting to an external power supply.

13. The oral utensil disinfection box according to claim 12, wherein the circuit board is positioned above a disinfection cavity, and the battery is positioned on one side of the air duct away from the circuit board.

14. The oral utensil disinfection box according to claim 13, further comprising a display electrically connected to the control module, wherein the display is positioned on one side of the air duct away from the battery, the main body is provided with a first transparent cover plate at a position corresponding to the display, and a power output end of the driving unit is positioned between the first transparent cover plate and the disinfection cavity.

15. The oral utensil disinfection box according to claim 14, wherein the sensing unit is an infrared sensor; and the sensing unit is arranged on the circuit board and positioned below the display.

16. The oral utensil disinfection box according to claim 1, further comprising an adapter plate arranged inside the main body, wherein the sterilization and disinfection unit is arranged on the adapter plate, a total number of the disinfection cavities is multiple, the sterilization and disinfection units and the disinfection cavities are consistent in number, the main body is provided with a second opening at a position corresponding to the disinfection cavity, and each of the sterilization and disinfection units is placed in the second opening on each of the disinfection cavities.

17. The oral utensil disinfection box according to claim 1, wherein a bottom portion of the main body is provided with a hanging opening at a position corresponding to the disinfection cavity for avoiding a toothbrush handle, the main body is provided with an elastic clamping block on a side wall of the hanging opening, and the elastic clamping block has elasticity and is used for limiting the toothbrush handle at the hanging opening.

18. The oral utensil disinfection box according to claim 17, further comprising a U-shaped second position limiting member, wherein the second position limiting member is positioned below the disinfection cavity; the disinfection cavities and the hanging openings are both two in number, the second position limiting member comprises a connecting rod and two clamping rods, the connecting rod is arranged on the main body, the two clamping rods are connected to the connecting rod and are arranged at intervals, the two clamping rods are positioned between the two hanging openings, each of the clamping rods is equipped with the elastic clamping block, and the elastic clamping block is partially placed inside the hanging opening in an initial state.

19. The oral utensil disinfection box according to claim 1, wherein the box cover comprises a cover body and a second transparent cover plate, the second transparent cover plate is provided on the cover body for a user to observe the disinfection cavity, and the second transparent cover plate corresponds to a position of the first opening when the box cover is in the closed state.

20. The oral utensil disinfection box according to claim 19, wherein a third opening is defined in the cover body, a surface of the cover body is recessed at a periphery corresponding to the third opening to form an embedding slot, and the cover body is provided with a second engaging opening on a bottom surface corresponding to the embedding slot; the second transparent cover plate is provided with a second engaging block, and the second engaging block is engaged with the second engaging opening.
